(19) United States
(12) Patent Application Publication
(10) Pub. No.: US 2009/0030310 A1
(43) Pub. Date: Jan. 29, 2009

(54) Diagnostic probe for combining positron emission measurements with ultrasonography

(75) Inventors: James J. Hamill, Knoxville, TN (US); David D. Faul, Knoxville, TN (US); William Curtis Howe, Knoxville, TN (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 11/881,342

(22) Filed: Jul. 26, 2007

(51) Int. Cl. A61B 6/00; A61B 8/00

(52) U.S. Cl. 600/437; 600/407

(57) ABSTRACT

A system for dual-mode medical imaging is provided. The system features components for PET imaging as well as for ultrasonic imaging, with an internal probe that has components to provide capability for both. The system provides cost-efficient PET imaging for smaller regions and organs of interest than conventional full body PET scanner apparatus.

### TECHNICAL FIELD

In general, the invention relates to nuclear medical imaging. More particularly, the invention relates to dual mode image acquisition using positron emission tomography (PET) scanning and ultrasonography.

### BACKGROUND OF THE INVENTION

PET imaging systems have been known and commercially available for many years. PET is currently one of the most effective ways to diagnose cancer recurrences, metastases of cancer, whether an early stage of cancer is present or not, and, if cancer has spread, its responding to treatment. PET is also used in diagnosing certain cardiovascular and neurological diseases by highlighting areas with increased, diminished, or no metabolic activity.

A normal PET scanner's ability to image large regions of the body efficiently is directly due to its large imaging field of view. This is appropriate since the ability to do a PET study of the whole body is at present the major use of PET. However, when the clinical need is to image just a small region of the body, a normal PET scanner is larger and more expensive than is actually needed. (Examples of small regions of clinical interest include the prostate when there is a suspicion of cancer, plaque deposits near the arteries which are to be evaluated for inflammation, and the myocardium of the heart, whose perfusion with blood needs to be evaluated and whose viability is of concern.) Therefore, there is a need for a smaller PET scanner system that is less expensive and better suited for performing PET imaging of selectable, small regions of the body.

### SUMMARY OF THE INVENTION

According to the invention, a system for dual-mode medical imaging is provided which permits imaging of relatively small-scale regions of interest. The system features components for PET imaging as well as for ultrasonic imaging, with an internal probe that has components to provide capability for both.

Other aspects and features of the invention will be evident from reading the following detailed description of the preferred embodiments, which are intended to illustrate, not limit, the invention.

### BRIEF DESCRIPTION OF THE DRAWINGS

The invention will become more clearly understood from the following detailed description in connection with the accompanying drawings, in which:

FIG. 1 is a perspective view of components of a dual-mode imaging system according to the invention;

FIGS. 2A and 2B are perspective views of the probe shown in FIG. 1 illustrating the dual ultrasonic and PET imaging modalities;

FIG. 3 is a schematic section view illustrating the system shown in FIG. 1 in use to image a prostate;

FIG. 4 is a schematic view illustrating the use of a vascular embodiment of a dual imaging mode probe according to the invention; and

FIG. 5 is a schematic view of an imaging probe similar to that shown in FIGS. 1, 2A, and 2B but having multiple radiation (PET) detectors.

### DETAILED DESCRIPTION OF THE INVENTION

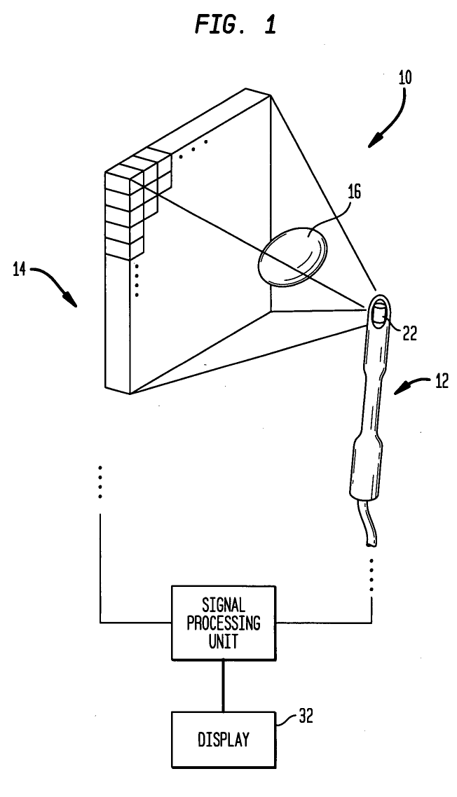

An imaging system 10 according to the invention is illustrated in FIG. 1. The imaging system includes a probe 12 that is sized and configured to be introduced into a patient's body and an external PET detector 14. The external PET detector 14 is generally conventional in that it is essentially a pixelated array of gamma ray-sensitive material such as lutetium oxyorthosilicate (LSO) or bismuth germanate (BGO), as is known in the art. The external detector 14 is, however, considerably smaller than PET detectors otherwise generally used in the art since, as illustrated and explained more fully below, it is intended to be used in much closer proximity to the patient's body and therefore need not be as expansive to register gamma photons generated upon annihilation of positrons emitted by an organ of interest 16 (e.g., the prostate). The acquired image data from the probe 12 and external detector 14 are processed in a signal processing unit (which can use well-known image signal processing techniques and therefore will not be further described here) to develop image signals that can be stored in a memory and/or sent to a display 32, which may be any suitable, known display device such as an LCD, LED, plasma discharge, CRT, etc.

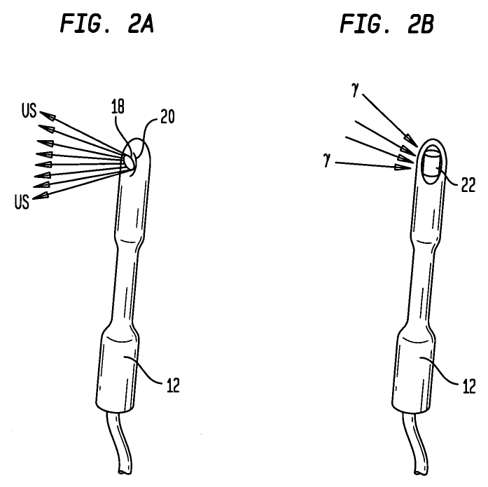

As illustrated in FIGS. 2A and 2B, the probe 12 is configured for dual imaging modalities. More particularly, as illustrated in FIG. 2A, the probe 12 includes an ultrasonic emitter 18 and an ultrasound receiver/transducer 20 in the head of the probe. Additionally, as illustrated in FIG. 2B, the probe 12 further includes a nuclear radiation detector 22 in the head of the probe. Like the external PET detector 14, the radiation detector 22 is sensitive to gamma radiation emitted upon annihilation of positrons emitted by the organ of interest. Unlike the external PET detector 14, however, the radiation detector 22 in the probe 12 consists of just a single crystal (essentially a large “pixel”). This eliminates the need to know a specific position where the gamma radiation strikes the radiation detector in order to generate an image (as is the case when using two pixelated detector arrays), although it does reduce the imaging volume to a cone beam as shown in FIG. 1 (as opposed to a cube, as can be imaged using two square pixelated detector arrays).

As a result, the imaging system 10 of the invention does not permit true tomography and does not have high enough resolution for whole-body applications; however, for imaging small regions that are close in to the organ of interest, the system provides acceptable imaging capabilities. Furthermore, because the detectors 14 and 22 of the imaging system 10 of the invention are used in very close proximity to the organ of interest 16—with probe detector 22 being placed internally (such as in the rectum) and therefore as close as possible to the organ of interest 16—photon attenuation by the patient's body is minimized. Moreover, the smaller size of the device over prior art PET detectors significantly reduces its cost.

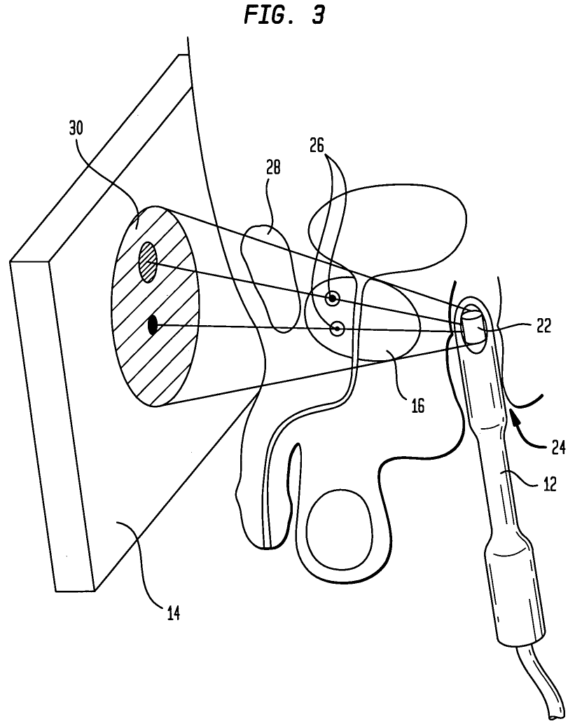

Exemplary use of the imaging system 10 according to the invention—e.g., for prostate cancer detection—is illustrated in FIG. 3. First, radiation activity is introduced (e.g., injected) into the organ of interest. As is known, cancerous tissue (e.g., masses 26) will take up certain radiopharmaceutical tracer substances at a faster rate than non-cancerous tissue and therefore will exhibit a greater concentration of the material than non-cancerous tissue, and that increased concentration can be measured by recording events that occur simultaneously, i.e. in coincidence, in the detectors 14 and 22, and incrementing the histogram image corresponding to the detector array 14. As shown, the head of the probe 12 is inserted into the patient's rectum 24, and the ultrasonic imaging capability of the probe 12 is used to locate the prostate (organ of interest) 16 and position the head of the probe generally near it. The external PET detector 14, on the other hand, is brought close to the patient's body generally located opposite the organ of interest from the probe detector 22, e.g., pressed against the pubic bone 28. The external PET detector 14 and probe radiation detector 22 are then used for positron imaging, which may take up to several minutes.

As noted above, the system of the invention does not permit true (i.e., three-dimensional) tomography. However, the system will produce two-dimensional projection images of the organ of interest, as indicated by the projection 30 shown against the external PET detector 14 in FIG. 3. By repositioning the external PET detector 14 and/or the probe radiation detector 22 to the extent possible, multiple two-dimensional projection images may be acquired, from which the medical practitioner may gauge the nature and extent of any unusual tissue masses. Once the PET-based images have been constructed, they may be displayed (e.g., on display device 32, FIG. 1) superimposed with ultrasonically generated images. The superposition allows the collection of additional diagnostic information and facilitates the taking of biopsies.

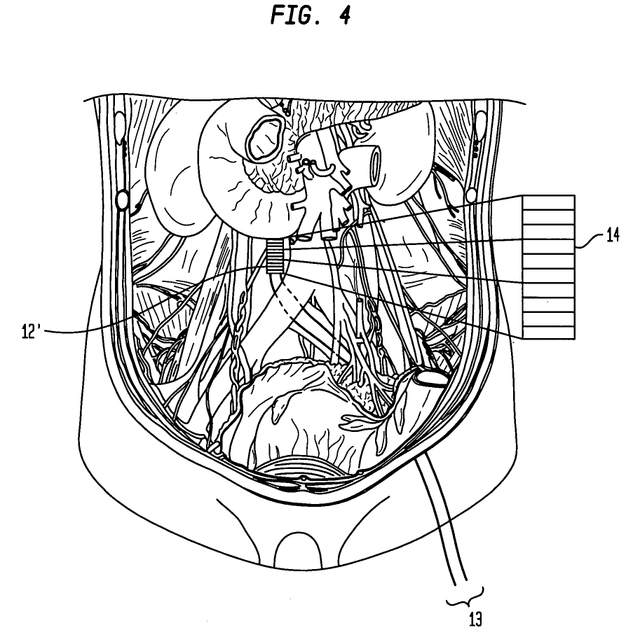

In the system 10 illustrated in FIGS. 1-3, the probe 12 is sized to be inserted into the patient's body via a natural orifice (e.g., the rectum). It is, however, possible to manufacture the probe considerably smaller, so as to be introducible into the body via an incision. For example, as illustrated in FIG. 4, a probe 12′ can be manufactured that is on the order of a few millimeters in length and diameter, with electrical signal leads 13. This configuration permits the probe 12 to be introduced into the body percutaneously. Thus, as illustrated in FIG. 4, the probe 12′ can be introduced into the femoral vein and moved into position to provide images of plaque deposits around the descending aorta. Further, the probe could be deployed via endoscope, to permit use in transesophageal echocardiography, the staging of gastrointestinal tumors, or other applications.

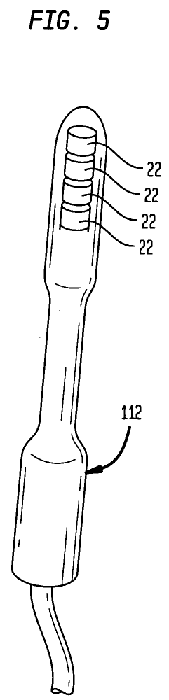

In an alternate embodiment shown in FIG. 5, a probe 112 includes multiple radiation detectors 22 (e.g., four as shown). As illustrated, the detectors 22 are aligned along the axis of the head of the probe 112, although other arrangements of the detectors are possible. Such a multiple-detector probe is advantageous for several reasons. First, it provides increased sensitivity as compared to the probes illustrated in FIGS. 1-3. Second, it facilitates limited-angle longitudinal tomographic reconstruction of the projections, thus creating images with partial depth information. This procedure is also known as digital tomosynthesis.

The foregoing description is meant to be illustrative of the invention and not limiting. Various modifications to the disclosed embodiments will occur to those having skill in the art. The scope of the inventory is defined by the following claims.

What is claimed is:

1. A system for imaging the internal organs of a patient, comprising:
an external PET detector comprising a pixelated array of gamma radiation-sensitive material;
an internal probe sized and configured to be inserted into the patient's body, the internal probe comprising an ultrasonic emitter and at least one detector formed from gamma radiation-sensitive material; and
a coincidence processor that receives and processes gamma event signals from said external PET detector and said internal probe.

2. The system of claim 1, wherein the internal probe is sized and configured to be inserted into the patient's rectum.

3. The system of claim 1, wherein the internal probe is sized and configured to be inserted into the patient's vascular system.

4. The system of claim 1, wherein the internal probe comprises multiple detectors formed from gamma radiation-sensitive material.

5. A probe for use in medical imaging, comprising a probe body with an ultrasonic transducer and at least one gamma radiation-sensitive detector disposed therein.

6. The probe of claim 5, wherein the probe is sized and configured to be inserted into a patient's body.

7. The probe of claim 6, wherein the probe is sized and configured to be inserted into the patient's rectum.

8. The probe of claim 6, wherein the probe is sized and configured to be inserted into the patient's vascular system.

9. The probe of claim 5, wherein the probe comprises multiple gamma radiation-sensitive detectors disposed therein.

10. A method of imaging an internal region of interest in a patient, comprising;
introducing a radiopharmaceutical tracer substance into the patient's body, the radiopharmaceutical tracer substance being adapted to be taken up by the region of interest;
inserting a probe having ultrasonic and PET imaging capabilities into the patient's body and, using the probe's ultrasonic imaging capabilities, positioning at least a portion of the probe in the vicinity of the region of interest;
disposing a PET detector array generally adjacent to the patient's body and, using the PET detector array and the probe's PET imaging capabilities, acquiring one or more PET images of the region of interest.

11. The method of claim 10, wherein the probe comprises multiple gamma radiation-sensitive detectors disposed therein.

12. The method of claim 10, wherein the probe is inserted into the patient's rectum.

13. The method of claim 10, wherein the probe is inserted into the patient's vascular system.
